(19) United States
(12) Patent Application Publication
(10) Pub. No.: US 2025/0195867 A1
(43) Pub. Date: Jun. 19, 2025

(54) BLOOD PUMP MOTOR

(71) Applicants: ABIOMED, Inc., Danvers, MA (US); Abiomed Europe GmbH, Aachen (DE)

(72) Inventors: Frank KIRCHHOFF, Aachen (DE); Jim Po WANG, Aachen (DE)

(73) Assignees: ABIOMED, Inc., Danvers, MA (US); Abiomed Europe GmbH, Aachen (DE)

(21) Appl. No.: 18/976,924

(22) Filed: Dec. 11, 2024

Related U.S. Application Data
(60) Provisional application No. 63/610,689, filed on Dec. 15, 2023.

(51) Int. Cl. A61M 60/416; A61M 60/13; A61M 60/17; A61M 60/216

(52) U.S. Cl. CPC A61M 60/416; A61M 60/13; A61M 60/17; A61M 60/216

(57) ABSTRACT

A blood pump motor may be provided. The blood pump motor may have a rotor, and a stator disposed around at least a portion of the rotor. The rotor may have a central axis of rotation. The rotor may include a magnet having a length extending from a distal end to a proximal end. The rotor may be free of an opening extending from the distal end to the proximal end, and/or the magnet may have a distal end portion, a proximal end portion, and a rotationally symmetrical middle portion located between the distal end portion and the proximal end portion, the middle portion having a circular cross-section.

### CROSS-REFERENCE TO RELATED APPLICATIONS

The present application claims priority to U.S. Provisional Patent No. 63/610,689, filed Dec. 15, 2023, the contents of which are incorporated by reference herein in its entirety.

### TECHNICAL FIELD

The present application is drawn to blood pumps, and specifically to an improved blood pump motor.

### BACKGROUND

To function as part of a medical device, blood pumps must be capable of moving a large amount of blood from one location to another. In many instances, a patient will benefit when a medical procedure uses a blood pump having as small in diameter as possible. However, as diameters decrease, the torque generated by conventional blood pump motors decreases. At a certain point, that torque is simply too low to function as an effective blood pump.

### BRIEF SUMMARY

In various aspects, a blood pump motor may be provided. The blood pump motor may include a rotor having a central axis of rotation. The rotor may include a magnet having a length extending from a distal end to a proximal end. The motor may include a stator disposed around at least a portion of the rotor. A diameter of the blood pump motor may be less than 3.3 mm.

Further, (i) the rotor may be free of an opening extending from the distal end of the magnet to the proximal end of the magnet, and/or (ii) the magnet may have a distal end portion, a proximal end portion, and a rotationally symmetrical middle portion located between the distal end portion and the proximal end portion, the middle portion having a circular cross-section.

In some embodiments, the blood pump motor may include a housing disposed around the stator. The housing may be composed of a metal alloy. The metal alloy may include iron, chromium, and aluminum.

In some embodiments, the proximal end portion and/or the distal end portion may include a bore extending into an outer surface, or may include a protrusion extending away from an outer surface. In some embodiments, at least one bore or protrusion may be configured to extend radially inward or outward from the outer surface. In some embodiments, at least one bore or protrusion may be configured to extend axially inward or outward from the outer surface.

In some embodiments, a distal shaft may be operably coupled to the distal end of the magnet, a proximal shaft may be operably coupled to the proximal end of the magnet, or both. A distal bore may extend partially into a distal end of the magnet, a proximal bore may extend partially into a proximal end of the magnet, or both. The distal bore may extend through the distal end portion and the middle portion may be free of the distal bore, the proximal bore may extend through the proximal end portion and the middle portion may be free of the proximal bore, or both. The distal bore may form a chamfered surface at the distal end of the magnet, the proximal bore may form a chamfered surface at the proximal end of the magnet, or both. The distal shaft may extend into the distal bore and the distal bore may extend less than 3 mm into the magnet. The proximal shaft may extend into the proximal bore and the proximal bore may extend less than 3 mm into the magnet, or both. In some embodiments, at least a portion of the distal shaft and/or the proximal shaft may have a non-circular cross-section (such as a geometric shape having 3-10 sides, or having one or more recesses, protrusions, or both) when viewed along its central axis.

In some embodiments, the distal shaft may be coupled to a distal shaft cap at a proximal end of the distal shaft, and/or the proximal shaft may be coupled to a proximal shaft cap at a distal end of the proximal shaft. The distal shaft cap and/or proximal shaft cap may have an inner surface configured to be coupled to an outer surface of the magnet. At least a portion of the inner surface of the distal shaft cap and/or proximal shaft cap may have a non-circular cross-section (such as a geometric shape having 3-10 sides, or having one or more recesses, protrusions, or both) when viewed along its central axis. The distal shaft cap and/or proximal shaft cap may include a metal, a ceramic, or both. The distal shaft and/or proximal shaft may be adhered to the magnet. In some embodiments, the distal shaft and/or proximal shaft may be mechanically coupled to the magnet.

In some embodiments, the distal shaft and/or proximal shaft may be integral with the magnet. The magnet may include an integral distal shaft extending from the distal end of the magnet, an integral proximal shaft operably coupled to the proximal end of the magnet, or both. In some embodiments, both the distal shaft and the proximal shaft may be operably coupled to the magnet.

In some embodiments, the distal shaft may be longer than the proximal shaft. In some embodiments, the distal shaft may be the same length as the proximal shaft. In some embodiments, the distal shaft may be shorter than the proximal shaft.

In some embodiments, a distal bearing may be disposed distal to the magnet, a proximal bearing may be disposed proximal to the magnet, or both. The proximal shaft may extend partially into the proximal bearing. The distal shaft may extend through the entire distal bearing. A thrust washer may be disposed between the magnet and the distal bearing.

In various aspects, a blood pump may be provided. The blood pump may include a motor section having a blood pump motor as disclosed herein. The blood pump may include a pump housing attached to the motor section. The pump housing may define an input port and an output port. The blood pump may include an impeller disposed within the pump housing. The impeller may be configured such that when the impeller rotates, it causes to pump blood from the input port to the output port. The impeller may be operably coupled to the blood pump motor. In some embodiments, the impeller may be coupled to a distal shaft of the blood pump motor. The impeller may be alternatingly radially compressible and radially expandable.

The blood pump may include a mesh filter configured to be mounted over an exterior of the pump housing. The mesh filter may include a polyurethane.

In some embodiments, the pump housing may include a plurality of generally helical first struts wound about the longitudinal axis and a plurality of second struts, the plurality of generally helical first struts and the plurality of second struts collectively defining a plurality of apertures therebetween. The pump housing may be alternatingly radially compressible and radially expandable. The plurality of generally helical first struts and the plurality of second struts may be absent any circumferential, relative to a longitudinal axis of the pump housing, struts.

The blood pump may include a catheter. The catheter may be operably coupled to a distal end of the motor section. The blood pump may include a controller. The controller may be in electrical communication with the blood pump motor.

### BRIEF DESCRIPTION OF DRAWINGS

The accompanying drawings, which are incorporated in and constitute a part of this specification, illustrate embodiments of the present invention and, together with a general description of the invention given above, and the detailed description of the embodiments given below, serve to explain the principles of the present invention.

FIG. 1 is depiction of a blood pump laid through the aorta, extending through the aortic valve into the left ventricle.

FIG. 2 is an illustration of a cross-sectional view of an embodiment of a blood pump motor.

FIG. 3A is an illustration of a cross-sectional view of an embodiment of a magnet.

FIG. 3B is an illustration of a magnified cross-sectional view of section A of FIG. 3A and FIG. 5.

FIGS. 4-5 are illustrations of cross-sectional views of embodiments of a magnet.

FIGS. 6A-6D are illustrations of embodiments of cross-sectional views of a magnet from cutting plane B of FIG. 5.

FIG. 7 is an illustration of a cross-sectional view of an embodiment of a magnet with distal and proximal shaft caps.

FIGS. 8A-8E are illustrations of embodiments of cross-sectional views of a magnet and a shaft cap from cutting plane C of FIG. 7.

FIG. 9 is an illustration of a perspective view of a magnet.

FIG. 10 is an enlarged side cut-away views of an expandable housing of an embodiment of a blood pump in an expanded state.

FIG. 11 shows a perspective view of an expandable filter formed of a mesh of filaments and mounted on a distal end region of the expandable housing of an embodiment of a blood pump.

FIG. 12 is a side view, of the expandable filter formed of a filter tube.

FIG. 13 is an illustration of a cross-sectional view of an embodiment of a blood pump motor.

It should be understood that the appended drawings are not necessarily to scale, presenting a somewhat simplified representation of various features illustrative of the basic principles of the invention. The specific design features of the sequence of operations as disclosed herein, including, for example, specific dimensions, orientations, locations, and shapes of various illustrated components, will be determined in part by the particular intended application and use environment. Certain features of the illustrated embodiments have been enlarged or distorted relative to others to facilitate visualization and clear understanding. In particular, thin features may be thickened, for example, for clarity or illustration.

### DETAILED DESCRIPTION

The following description and drawings merely illustrate the principles of the invention. It will thus be appreciated that those skilled in the art will be able to devise various arrangements that, although not explicitly described or shown herein, embody the principles of the invention and are included within its scope. Furthermore, all examples recited herein are principally intended expressly to be only for illustrative purposes to aid the reader in understanding the principles of the invention and the concepts contributed by the inventor(s) to furthering the art and are to be construed as being without limitation to such specifically recited examples and conditions. Additionally, the term, “or,” as used herein, refers to a non-exclusive or, unless otherwise indicated (e.g., “or else” or “or in the alternative”). Also, the various embodiments described herein are not necessarily mutually exclusive, as some embodiments can be combined with one or more other embodiments to form new embodiments.

The numerous innovative teachings of the present application will be described with particular reference to the presently preferred exemplary embodiments. However, it should be understood that this class of embodiments provides only a few examples of the many advantageous uses of the innovative teachings herein. In general, statements made in the specification of the present application do not necessarily limit any of the various claimed inventions. Moreover, some statements may apply to some inventive features but not to others. Those skilled in the art and informed by the teachings herein will realize that the invention is also applicable to various other technical areas or embodiments.

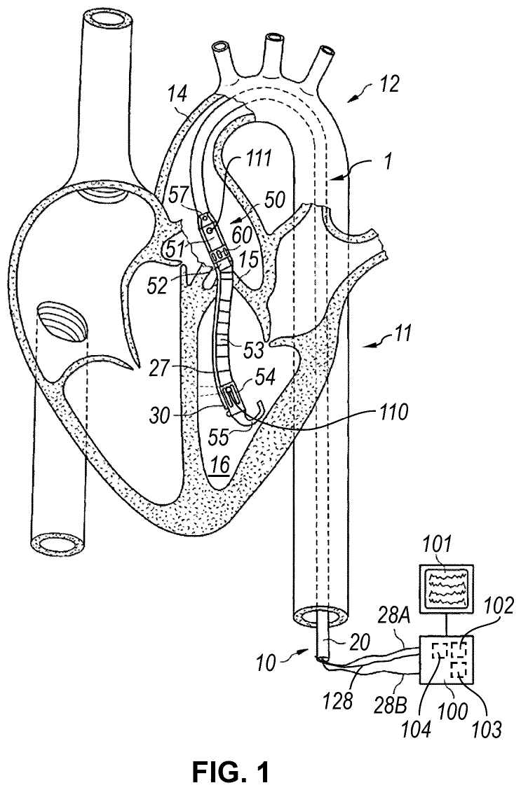

Turning now to the figures, FIG. 1 shows an exemplary blood pump 1 having a catheter 10 which may be introduced into a patient's heart. For example, as shown in this figure, the pump may be inserted into the descending aorta 11 retrograde in some embodiments. As is known, the descending aorta is part of the aorta 12 which first ascends from the heart and then descends and has the aortic arch 14. At the beginning of the aorta 12 there is located the aortic valve 15 which connects the left ventricle 16 to the aorta 12 and through which the intravascular blood pump may extend.

As will be appreciated, blood pumps may be inserted into other suitable portions of the body. For example, in some embodiments, a blood pump may be inserted into a right ventricle. In some embodiments, a blood pump may be inserted into a femoral artery or a jugular vein.

As shown in FIG. 1, the blood pump 1 may include a rotary pumping device 50 which may be coupled to a distal end of the catheter hose 20 and having a pump section 52 (sometimes referred to herein as simply “pump”) disposed at an axial distance therefrom. In some embodiments, the blood pump may include a motor section 51 operably coupled to the pump section. In some embodiments, the motor section is positioned distally from the distal end of the catheter hose.

The blood pump may include a cannula 53 (which may be a flexible flow cannula) protruding in the distal direction from the inflow end of the pump section 52 and having a suction inlet 54 located at its end. As FIG. 1 shows, the catheter exits from the catheter hose 20 at an exit point 57 and is guided along the flexible flow cannula. Distally of the suction inlet 54 there is provided a soft-flexible tip 55, which can be configured for example as a “pigtail” or in a J shape. Through the catheter hose 20 there extend different lines and devices which may be important for operating the pumping device 50.

In some embodiments, the catheter may include a sliding tube 27 in which various wires or fibers 28A, 28B may be freely movable. The various wires or fibers may be operably coupled to a controller 100. In some embodiments, the optical fibers 28A, 28B may be respectively part of an optical sensor (such as a pressure sensor) whose sensor heads 30 and 60 may be located in the vicinity of the suction inlet 54, on the one hand, and on the outside on the housing of the pump section 52, on the other hand. FIG. 1 shows sensor head 30 as being on the outside of the blood pump, but the sensor head could also be located internal to the blood pump, preferably within an inflow cage, the inflow cage having lumens therethrough that define the suction inlet 54. In such embodiments, information (e.g., pressure information) may be transmitted from the pump to the controller 100 and may be converted into electrical signals in the controller 100 and displayed e.g., on a display screen 101.

As also shown in FIG. 1, the pump may include one or more sensors 110. As shown in this figure, the sensor 110 may be placed on the inlet 54 housing. In some embodiments, the sensor also may be positioned on other suitable portions of the pump, such as on the flexible tip 55, the cannula 53, and/or on the outlet 60 housing. As will be appreciated, in some embodiments, the pump may include only a single such sensor. However, in some embodiments, the pump may include more than one such sensor (e.g., both the sensor 110 as well as an additional sensor 111 arranged for spectroscopy-based sensing). If a plurality of sensors are used, two or more sensors may be located at the same portion of the pump (e.g., on the inlet housing) and/or on different portions of the pump (e.g., on the flexible tip and the inlet housing). As also shown in FIG. 1, one or more fibers (e.g., first fiber 128) may extend between the sensor 110 and the controller 100.

The controller may include one or more processors 102 that may, individually or collectively, control operation of the pump, and optionally perform other functions. As will be understood by those of skill in the art, the processor(s) may be coupled to various other components 103, 104, such as memory, non-transitory computer-readable storage medium, a wired or wireless interface, etc., as needed to perform various functions.

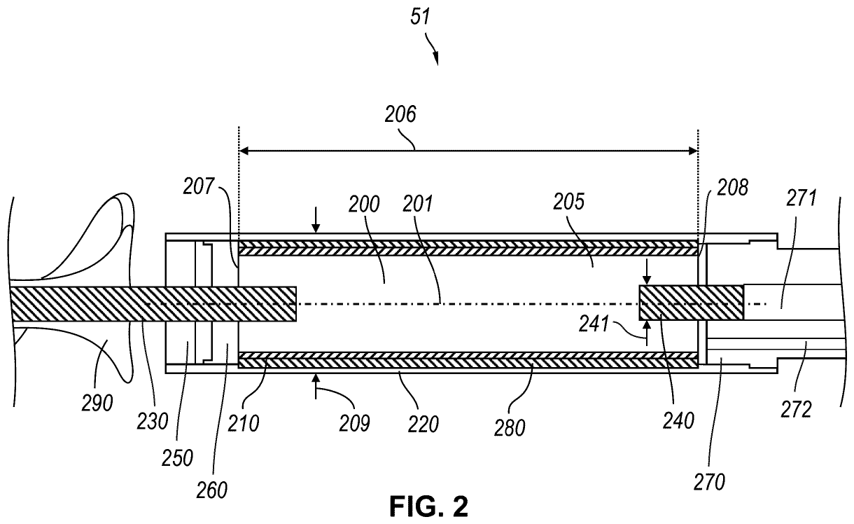

Referring to FIG. 2, a cross-sectional view of a blood pump motor (e.g., a motor section 51) can be seen. The blood pump motor may include a rotor 200 having a central axis 201 of rotation. The rotor may include a magnet 205 having a length 206 extending from a distal end 207 to a proximal end 208. The magnet may be comprised of any appropriate magnetic material. In some embodiments, the magnet may be comprised of niobium, iron, and boron.

A magnet having any appropriate length may be utilized. In some embodiments, the length may be at least about 12 mm. In some embodiments, the length may be at least about 13 mm. In some embodiments, the length may be at least about 14 mm. In some embodiments, the length may be at least about 15 mm. In some embodiments, the length may be at least about 16 mm. In some embodiments, the length may be no more than about 16 mm. In some embodiments, the length may be no more than about 17 mm. In some embodiments, the length may be no more than about 18 mm. In some embodiments, the length may be no more than about 19 mm. In some embodiments, the length may be no more than about 20 mm.

In a blood pump, magnets often are required to work in a corrosive environment, such as in a purging liquid flowing between the rotor and the stator. The purge fluid is typically an aqueous fluid, possibly a fluid containing chloride. Chloride is highly corrosive for rare earth metal based magnets, but also water and oxygen dissolved in the water cause severe corrosion within very short time spans of only a few hours. Thus, magnets, especially rare earth metal based permanent magnets, such as neodymium iron boron magnets, may need to be protected against corrosion. Corrosion resistance may be improved by coating the magnets with protective coatings.

Usual coatings are nickel coatings and coatings based on epoxy resins, and, especially for blood pumps, titanium coatings and Parylene coatings (such as those Parylene C) are known. In some embodiments, the coating may have a thickness (e.g., the difference between an outer radius of the magnet and an outer radius of the coating) may be less than 0.5 mm. In some embodiments, the thickness may be less than 0.025 mm. In some embodiments, the thickness may be less than 0.015 mm. In some embodiments, the thickness may be less than 0.01 mm. In some embodiments, the thickness may be 0.005-0.01 mm.

The motor may include a stator 210 disposed around at least a portion of the rotor. A diameter 209 of the blood pump motor may be relatively small. In some embodiments, the diameter may be less than about 3.5 mm. In some embodiments, the diameter may be less than about 3.4 mm. In some embodiments, the diameter may be less than about 3.3 mm.

The blood pump motor may include a housing 220 disposed around the stator 210. The housing may be composed of a metal alloy. The metal alloy may include iron, chromium, and aluminum.

The motor may include a magnetic yoke 280 disposed around the stator 210. Preferably, the yoke is disposed between the stator and the housing. Any magnetically conductive material may be used to form the yoke (e.g., a “back iron”) of the electric motor. In this manner, the yoke may be used to close the magnetic flux of the electric motor. In some embodiments, the yoke may include cobalt. In some embodiments, the stator may have a thickness (e.g., a difference between an inner radius of the stator and an outer radius of the stator) of no more than 0.5 mm, such as 0.1-0.2 mm. In some embodiments, the yoke may have a thickness (e.g., a difference between an inner radius of the yoke and an outer radius of the yoke) of no more than 0.2 mm, such as 0.05-0.2 mm.

In some embodiments, the housing is formed of a material that allows the housing to function as a yoke, so no separate yoke is required. That is, in some embodiments, the motor may be free of a separate yoke. In these embodiments, the housing may be composed of a magnetically conductive material. The housing may be composed of an alloy. The alloy may include cobalt, iron, chromium, aluminum, or a combination thereof.

A distal shaft 230 may be operably coupled to the distal end 207 of the magnet. A proximal shaft 240 may be operably coupled to the proximal end 208 of the magnet. The shafts may have an outer diameter 241 that is no more than about 1 mm. In some embodiments, the shafts may have an outer diameter that is no more than about 0.9 mm.

The length of a distal and/or proximal shaft may be selected based on desired design characteristics. In some embodiments, a distal shaft 230 may be longer than a proximal shaft 240. In some embodiments, the distal shaft 230 may be the same length as the proximal shaft 240. In some embodiments, the distal shaft 230 may be shorter than the proximal shaft 240.

A distal bearing 250 may be disposed distal to the magnet. The distal bearing may be disposed at least partially within housing 220. The distal shaft may extend through the entire distal bearing 250. A thrust washer 260 may be disposed between the magnet 205 and the distal bearing 250. The distal shaft may extend through the entire thrust washer 260. Distal to the motor, an impeller 290 may be coupled to the distal shaft.

A proximal bearing 270 may be disposed proximal to the magnet. The proximal bearing may be disposed at least partially within housing 220. In some embodiments, the proximal bearing may include one or more channels 271, 272 extending through the bearing. The proximal bearing may include a channel 271 coaxial with the central axis 201. The proximal bearing may include a channel 272 offset from a central axis 201. The proximal shaft may extend partially into the proximal bearing. The proximal shaft may extend entirely through the proximal bearing. The proximal shaft may extend at least partially through the channel 271 coaxial with the central axis 201.

The magnet may be designed in various ways so as to improve torque of the motor and allow the torque to be transferred, e.g., to a shaft.

In some embodiments, the rotor may be free of an opening extending from the distal end 207 of the magnet to the proximal end 208. As seen in FIG. 2, a distal shaft 230 may be operably coupled to the distal end 207 of the magnet, a proximal shaft 240 may be operably coupled to the proximal end 208 of the magnet, or both.

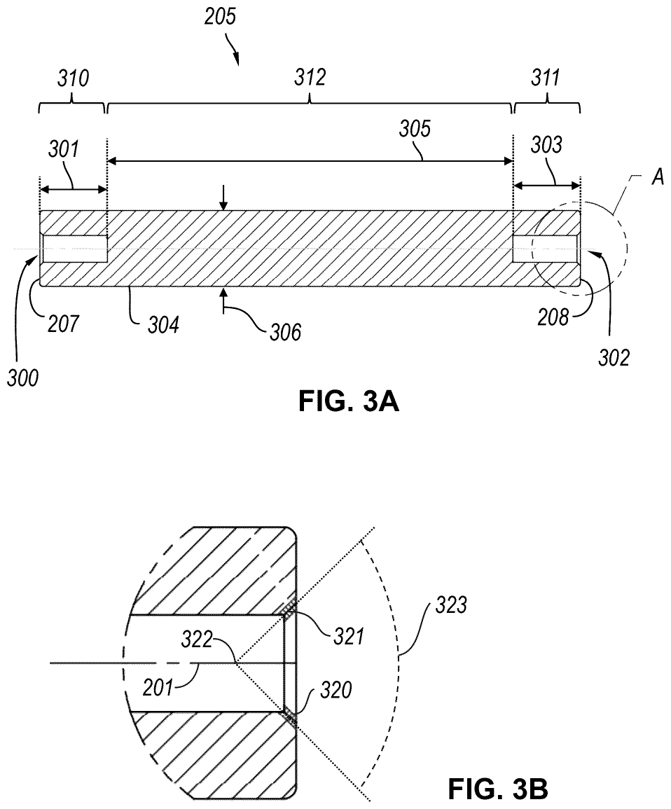

Referring to FIGS. 3A-3B, the magnet 205 may be understood as having three portions-a distal end portion 310, a proximal end portion 311, and a middle portion 312 extending from the distal end portion to the proximal end portion. In some embodiments, the middle portion may be considered the “solid” portion of the magnet; that is, the portion of the magnet that is free of any cap around it or shaft extending through it.

The distal end portion may include a bore 300 extending into an outer surface 304. As used herein, the term “bore” is not restricted to holes generated by drilling. Rather, the term is intended to any hole or cavity extending from an outer surface of the magnet and having a single opening. That is, the hole or cavity only extends partially through the magnet.

The proximal end portion may include a bore 302 extending into an outer surface 304. As seen, at least one bore may be configured to extend axially inward (e.g., in a direction parallel to a central axis 201 of the magnet) from the outer surface. As will be recognized, however, in some embodiments, one or more bores may extend radially inward from the outer surface. As will be recognized, in some embodiments, one or more bores may extend radially outward from the outer surface. The distal bore 300 may extend partially into a distal end 207 of the magnet. A proximal bore 302 may extend partially into a proximal end 208 of the magnet. The distal bore may extend through the distal end portion and the middle portion may be free of the distal bore. The proximal bore may extend through the proximal end portion and the middle portion may be free of the proximal bore.

In some embodiments, the distal shaft may extend a distance 301 into the distal bore. In some embodiments, the proximal shaft may extend a distance 303 into the proximal bore. In some embodiments, the distal and/or proximal bore may extend no more than about 3.5 mm into the magnet. In some embodiments, the distal and/or proximal bore may extend no more than about 3 mm into the magnet. In some embodiments, the distal and/or proximal bore may extend less than 3 mm into the magnet. In some embodiments, the distal and/or proximal bore may extend no more than about 2.5 mm into the magnet. In some embodiments, the distal and/or proximal bore may extend no more than about 2.25 mm into the magnet. In some embodiments, the distal and/or proximal bore may extend no more than about 2 mm into the magnet. In some embodiments, the distal bore and the proximal bore may extend an equal distance into the magnet. In some embodiments, the distal bore and the proximal bore may extend different distances into the magnet. In some embodiments the distances each bore extends into the magnet may be selected such that a length 305 of the middle portion, free of any bore, may be at least about 12 mm in length. In some embodiments, the length of the middle portion may be at least about 11 mm in length. In some embodiments, the length of the middle portion may be at least about 11.5 mm in length.

In some embodiments, the magnet may have a relatively small diameter 306. In some embodiments, the diameter may be less than about 3.3 mm.

The distal bore may form a chamfered surface at the distal end of the magnet, the proximal bore may form a chamfered surface at the proximal end of the magnet, or both. Referring to FIG. 3B, the chamfer 320 may have surfaces 321. An angle 323 may be formed from lines, parallel to the surfaces 321 and in a plane with the central axis 201, extending from a vertex 322. In some embodiments, the angle may be at least 80 degrees. In some embodiments, the angle may be at least 85 degrees. In some embodiments, the angle may be at least 90 degrees.

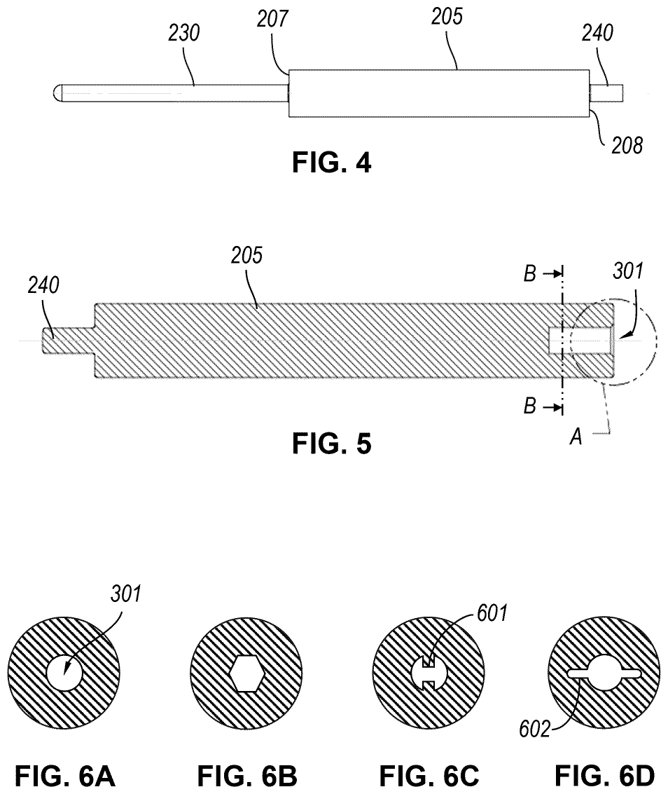

Rather than using bores, one or more shafts may be operably coupled to an end of the magnet. Referring to FIG. 4, the distal shaft 230 may be operably coupled to the distal end 207 and/or the proximal shaft 240 may be coupled to the proximal end 208 of the magnet. The distal shaft and/or the proximal shaft may be made of magnetic material. Referring to FIG. 5, in some embodiments, the distal shaft and/or proximal shaft may be integral with the magnet (here, proximal shaft 240 is shown as being integral with the magnet, while the distal end includes a distal bore configured to receive a distal shaft). That is, in some embodiments, the magnet may include an integral distal shaft extending from the distal end of the magnet, an integral proximal shaft operably coupled to the proximal end of the magnet, or both. The term “integral” generally refers to being made at the same time or being incapable of being separated without damaging one or more of the integral parts, such as an individual component not consisting of subcomponents connected in a bonded and frictional manner. In some embodiments, both the distal shaft and the proximal shaft may be operably coupled to the magnet.

As seen in FIGS. 6A-6D, the bore may be designed in various ways to interface with a portion of a shaft (either a distal or proximal shaft as appropriate). In some embodiments, the bore may have a circular cross-section (see FIG. 6A). In some embodiments, the bore may have a cross-section that defines a geometric shape. The geometric shape may have, e.g., 3-10 sides (such as the hexagon shown in FIG. 6B). In some embodiments, the bore may have a cross-section that defines a shape other than a geometric shape (such as an arbitrary shape). In some embodiments, the bore's cross-section may have one or more recesses 601 (see FIG. 6C), protrusions 602 (see FIG. 6D), or both.

The shafts may have a portion of the shaft configured to interact with the bores. As such, in some embodiments, at least a portion of the distal shaft and/or the proximal shaft may have a non-circular cross-section (such as a geometric shape having 3-10 sides, or having one or more recesses, protrusions, or both) when viewed along its central axis.

The distal shaft and/or proximal shaft may be adhered to the magnet. In some embodiments, the distal shaft and/or proximal shaft is mechanically coupled to the magnet. In some embodiments, the shafts may be coupled directly to the magnet. In some embodiments, the shafts may be coupled indirectly to the magnet.

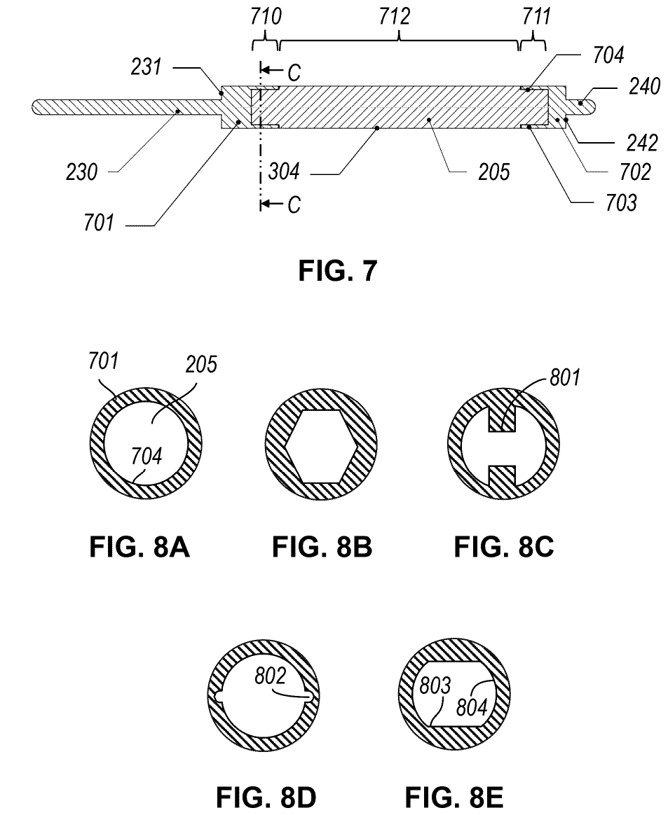

For example, referring to FIG. 7, the magnet 205 may be coupled to one or more caps. This may include a distal shaft cap 701 configured to be coupled to a distal end of the magnet, and a proximal shaft cap 702 configured to be coupled to a proximal end of the magnet. Each cap may include one or more materials. Each caps may, independently, include a magnetic material. Each caps may, independently, include a ceramic. Each caps may, independently, include a metal. Each caps may, independently, be configured such that a portion 703 of a cap extends over a portion of the magnet. For example, in some embodiments, similar to that described with bores, the magnet may have a distal end portion 710 surrounded by a portion of the distal shaft cap, the magnet may have a proximal end portion 711 surrounded by a portion of the proximal shaft cap, and there may be a middle portion 712 disposed between the distal end portion and the proximal end portion that is free from any shafts, and free from any shaft caps. The distal shaft 230 may be coupled to a distal shaft cap 701 at a proximal end 231 of the distal shaft. The proximal shaft 240 may be coupled to a proximal shaft cap 702 at a distal end 242 of the proximal shaft.

The caps may be configured to interface with a portion of the magnet. Each cap may, independently, be configured such that an inner surface 704 of the cap may be configured to be coupled to an outer surface 304 of the magnet.

In some embodiments, at least a portion of the inner surface of one or both caps may have a circular cross-section (see FIG. 8A). In some embodiments, at least a portion of the inner surface of one or both caps may have a non-circular cross-section. In some embodiments, at least a portion of the inner surface of one or both caps may have a cross section with a geometric shape having 3-10 sides (such as the hexagon shown in FIG. 8B). In some embodiments, at least a portion of the inner surface of one or both caps may have one or more recesses 801 (see FIG. 8C), protrusions 802 (see FIG. 8D), or both when viewed along its central axis. Referring to FIG. 8E, in some embodiments, at least a portion of the inner surface of one or both caps may have one or more flat surfaces 803 and one or more curved surfaces 804.

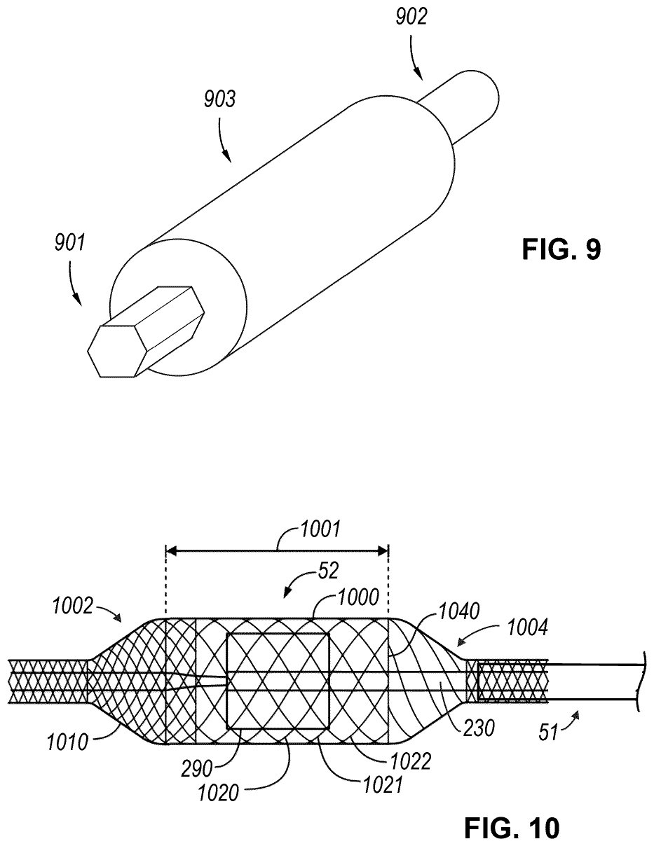

As seen from the above, it is clear that in some embodiments, the magnet may have a distal end portion 901, a proximal end portion 902, and a rotationally symmetrical middle portion 903 located between the distal end portion and the proximal end portion, the middle portion having a circular cross-section. Each end portion may, independently, have a circular cross-section, or have at least a portion having a non-circular cross-section. As seen in FIG. 9, in some embodiments, the entire end portion may have a non-circular cross-section.

In various aspects, a blood pump may be provided. Referring to FIG. 1, the blood pump 1 may include a motor section 51 having a blood pump motor as disclosed herein. The blood pump may include a catheter 20 operably coupled to a distal end of the motor section 51. The blood pump may include a controller 100 in electrical communication with the blood pump motor.

Referring to FIG. 10, the pump section 52 may include an impeller 290 disposed inside a pump housing 1000. The pump housing 1000 and the impeller 290 can, but need not necessarily, be expandable. The impeller may be operably coupled to the blood pump motor. The impeller may be mechanically coupled, via a flexible drive shaft (e.g., distal shaft 230) that extends to the motor section 51. The motor rotates the impeller, via the drive shaft, to cause blood (e.g., from a blood vessel) to flow from a blood flow inlet (input port) 1002 at a distal end of the pump section 108 to a blood flow outlet (output port) 1004 located proximal of the blood flow inlet 1002.

A filter 1010 may be disposed in fluid communication between: (a) the interior volume of a blood vessel (e.g., the left ventricle), external to the pump housing 1000, and (b) the input port 1002. Although the filter 1010 is described in relation to an expandable housing 1000 and impeller, the filter 1010 may also be used with a non-expandable housing 1000 and impeller.

The struts 1020-1021 that are used in the filter may be made of wire or other filament. As shown, the pump housing 1000 may provide a cage around the impeller 290. When radially expanded, the length 1001 of the housing 1000 may be less than the length when the housing may be radially compressed. The change in length may be due to unwinding of the struts 1020-1022, when the housing expands. In some embodiments, the change in length may be about 1-2 mm.

As is well-understood in the art, the expandable housing 1000, expandable impeller 290 and expandable filter 1010 may be kept in their compressed states by a suitable compression sleeve (not shown) slid over the expandable housing 1000, expandable impeller 290 and expandable filter 1010. The blood pump 1, with the expandable housing, expandable impeller, and expandable filter, may be transported through the patient's vascular system while the housing, impeller, and filter are in their compressed states. Once the pump section 52 is at its target location, the housing, the impeller, and the filter may be allowed to expand, e.g., by pushing the pump section out of the compression sleeve in a forward (distal) direction or by pulling back (in a proximal direction) the compression sleeve. With the compression sleeve removed, the housing expands, due to its shape-memory, superelastic or hyperelastic properties. At the same time, the impeller 290 expands due to its elasticity. As the housing 1000 expands radially away from the distal shaft 230, the housing 1000 may longitudinally contract to the length 1001. An inside central portion of the housing 1000 may have a sleeve or coating, which defines a channel, through which the blood may be pumped by the impeller 290. Proximally and distally of this channel, the housing 1000 may allow blood to be drawn into the housing 1000 and pushed out of the housing 1000, e.g., into an outflow cannula.

As is understood in the art, when the blood pump 1 may be in its expanded state and needs to be removed from the patient, the housing 1000 may be, e.g., pulled back into the compression cannula, which causes the housing 1000 to compress radially, and may cause the housing 1000 to longitudinally extend. The filter 1010 and the impeller 290 may also be compressed. The smaller diameter of the housing 1000 thus achieved facilitates removing the blood pump 1 from the patient through the vasculature. Thus, the pump housing 1000, the impeller 290 and the filter 1010 are each configured to be alternatingly radially compressed and radially expanded. Additional details of an expandable intravascular blood pump are provided in U.S. Pat. No. 8,439,859, the entire contents of which is hereby incorporated by reference herein, for all purposes.

FIG. 9 contains a perspective view of a distal section of the intravascular blood pump 1 with an intermediate tubular housing part 1100, a distal tapered housing part 1102 and a distal tubular housing part 1104. In this embodiment, the expandable filter 1010 may be a mesh filter made of filaments that are woven or connected to each other. Weaving is a method of production in which two distinct sets of filaments (warp and weft) are interlaced at angles to form a fabric. The warp is made up of longitudinal filaments, and the weft (or filling) is made up of lateral filaments. The way the warp and weft filaments interlace with each other is called the weave. The majority of woven products are created with one of three basic weaves: plain weave, satin weave or twill.

In plain weave, the warp and weft filaments cross at angles, aligned so they form a simple crisscross pattern. Each weft filament crosses the warp filaments by going over one, then under the next, and so on. The next weft filament goes under the warp threads that its neighbor went over, and vice versa. The filaments of a woven filter 1010 are preferably plain woven, although satin, twill or other weaves may be used. Preferably, the mesh is not knitted and contains no loops.

The satin weave may be characterized by four or more weft filaments floating over a warp filament, and four or more warp filaments floating over a single weft filament. Floats are missed interfacings, for example where the warp filament lies on top of the weft filaments in a warp-faced satin. The twill weave may be characterized by a pattern of diagonal parallel ribs. Twill weave may be made by passing the weft filament over one or more warp filaments, then under two or more warp filaments, and so on, with a “step,” or offset, between rows to create a characteristic diagonal pattern.

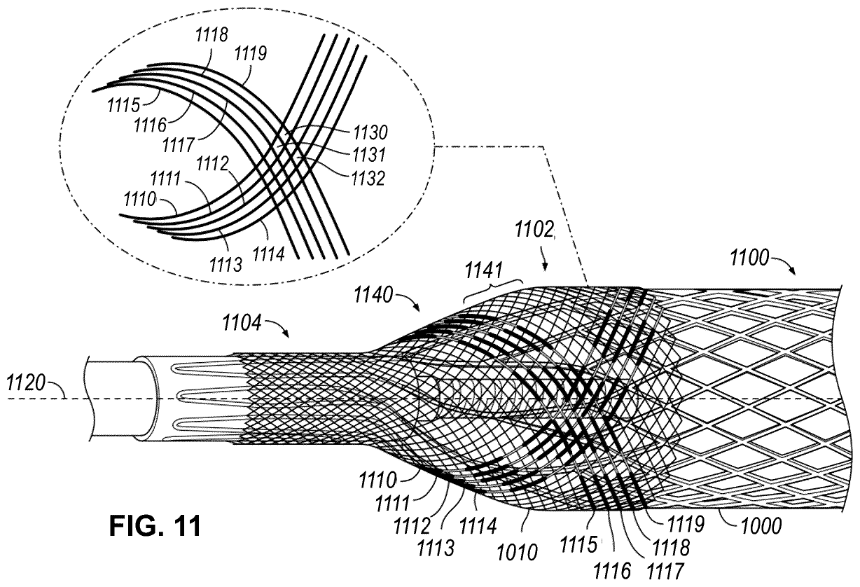

Referring to FIG. 11, the filter 1010 may be made of filaments, represented by filaments 1110-1119. The filaments 1110-1114 are generally helical first struts wound clockwise about a longitudinal axis 1120 of the pump housing 1000. As used herein, a “generally helical” curve is a generally smooth space curve. However, as used herein, pitch, radius, curvature and torsion may vary along the length of a helical curve. A helical curve may, but need not, wind more or less than 360° around an axis. Furthermore, a generally helical curve may include minor zigzags, not necessarily all the same. The mesh filter may be configured to be mounted over an exterior of the pump housing.

The filaments 1115-1119 may be generally helical second struts wound counterclockwise about the longitudinal axis 1120. The filaments 1110-1118 are indicated by heavy dashed lines, to make them easier to see in the drawing. These filaments 1110-1118 are also reproduced in an insert in FIG. 11, for clarity. The first struts 1110-1114 and the second struts 1115-1119 collectively define a plurality of apertures therebetween, represented by apertures 1130, 1131, and 1132. The first struts 1110-1114 and the second struts 1115-1119 are woven together, such that the plurality of apertures 1130-1132 is defined between respective adjacent first and second woven filaments 1110-1119.

The plurality of generally helical first struts and the plurality of second struts may be absent any circumferential, relative to a longitudinal axis of the pump housing, struts.

Each aperture of at least a subset of the plurality of apertures 1130-1132 may have a general rhombus or rhomboid or rectangular shape. As used herein, a rhomboid is a parallelogram in which adjacent sides are of unequal lengths and angles between adjacent sides are non-right angles. As used herein, a rhombus is a parallelogram in which adjacent sides are equal lengths and angles between adjacent sides are non-right angles. Rhomboids, rhombi and rectangles are not necessarily planar. Rhomboids, rhombi and rectangles may exist on curved surfaces, as exemplified by apertures 1130-1132. The sides of a rhomboid, a rhombus or a rectangle need not be perfectly straight, and the sides need not necessarily meet at corners, i.e., there may be a small radius where the two sides meet, for example as discussed in more detail below, with respect to corners in apertures defined by shaped foil tube filters.

In at least a middle portion 1141 of the tapered filter section 1140, the apertures 1130-1132 may be preferably approximately square shaped. As the diameter of the filter 1010 decreases, such as in the distal direction within the tapered filter section 1140, the apertures 1130-1132 may become progressively smaller, and the apertures may become rhomboid shaped, with their long axes extending longitudinally. At the smallest diameter of the tapered filter section 1140, the smaller inner angles of rhombus or rhomboid apertures may be less than about 75°.

As the diameter of the filter 1010 increases, such as in the proximal direction within the tapered filter section 1040, the apertures 1130-1132 may become progressively larger. At the largest diameter of the tapered filter section 1040, the larger inner angles of rhombus or rhomboid apertures may be greater than about 110°. The apertures may become rhomboid shaped, with their long axes extending circumferentially. These numbers correspond to an embodiment in which the larger diameter of the filter 1010 is about 2.5 times the smaller diameter of the filter 1010. For other ratios of large to small diameters of filter 1010, the angles can be adjusted.

The pump housing 1000 may be configured, when radially compressed, to longitudinally lengthen an amount that depends on an amount by which the pump housing 1000 is radially compressed. The filter 1010 may be configured, when radially compressed, to longitudinally lengthen an amount that depends on an amount by which the filter 1010 is radially compressed. The filter 1010 may be configured such that, for a given amount of radial compression, the filter 1010 and the pump housing 1000 longitudinally lengthen about equal amounts.

The filaments 1110-1119 may be a wire, such as Nitinol, suitable polymer, such as polyethylene terephthalate (PET) or PU, fiber or another suitable material. The filament 1110-1119 material is preferably a shape memory material. Individual filaments 1110-1119 may have a thickness of between about 10 μm and about 80 μm, or between about 20 μm and about 60 μm, such as about 40 μm. The catheter 20, the pump housing 1000, the impeller 290 and the filter 1010 are configured for use in a living patient, such that each aperture of the plurality of apertures 1130-1132 is sized to prevent ingestion, by the input port 1002, of heart tissue of the living patient.

In some embodiments where the filter 1010 is formed of a mesh, the mesh may be ironed (pressed under heat), prior to attaching the filter 1010 to the housing 1000. Such ironing may fuse crossing filaments 1110-1119, particularly if the filaments 1110-1119 are made of a suitable heat-fusible plastic. Such fused filaments 1110-1119 form a stronger mesh. The filter may be comprised of any appropriate material, preferably a polymeric material, such as a polyurethane.

In some embodiments, the woven fabric has a maximum distance between two adjacent filaments 1110-1119 of between about 0.3 mm (300 μm) and about 0.4 mm (400 μm), when the filter 1010 is in the expanded state. In some embodiments, each aperture of the plurality of apertures 1130-1132 has a largest dimension less than or equal to about 0.5 mm (500 μm), when the filter 1010 is in the expanded state. In some embodiments, each aperture of the plurality of apertures 1130-1132 has a largest dimension less than or equal to about 0.4 mm (400 μm), when the filter 1010 is in the expanded state. In some embodiments, each aperture of the plurality of apertures 1130-1132 has an area less than or equal to about 0.09 mm2, when the filter 1010 is in the expanded state. In some embodiments, each aperture of the plurality of apertures 1130-1132 has an area less than or equal to about 0.16 mm2, when the filter 1010 is in the expanded state.

As used herein, “largest dimension” includes a diagonal dimension, such as a dimension between two diagonally opposite corners of a quadrilateral. As used herein, “diameter” of a convex shape means a largest distance that can be formed between two opposite parallel lines tangent to the boundary of the convex shape. As used herein, “width” means the smallest such distance.

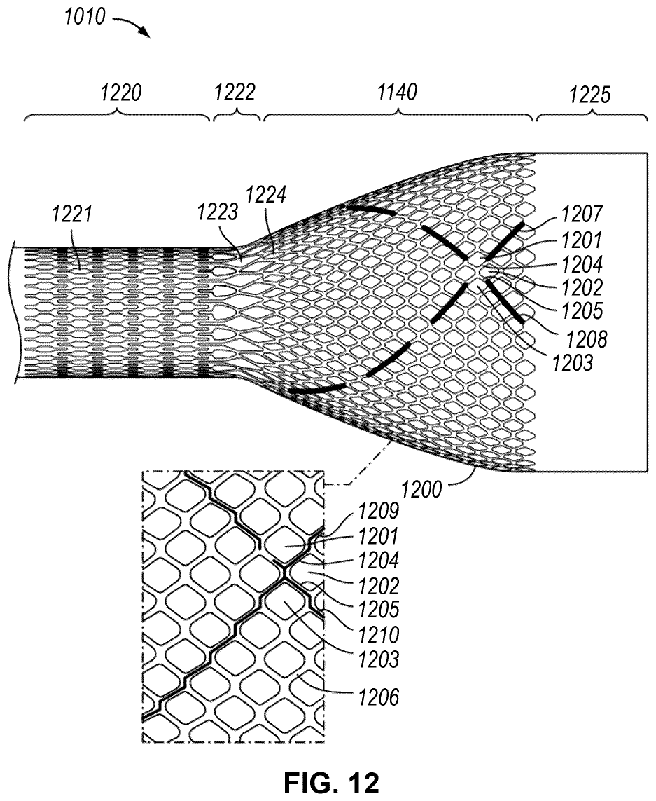

FIG. 12 is a side view of an expandable filter 1010 formed of a filter tube. In some embodiments, the tube may be a generally funnel-shaped tube. FIG. 12 includes an insert showing an enlarged portion of the expandable filter 1010. As noted, in some embodiments, the filter 1010 includes a shaped foil tube 1200 with the apertures. The apertures may be openings through a wall forming the tube. The wall may be, e.g., 10-100 μm thick. Examples of the apertures are shown at 1201, 1202, and 1203. An expandable filter 1010 made from a shaped foil tube 1200 may be compressed, i.e., radially made smaller, by folding some or all parts of the filter 1010. The filter 1010 may be expanded from its compressed state by unfolding the previously folded parts. Compression and expansion rely primarily on this folding and unfolding, rather than on elastic compression and elongation.

The apertures 1201-1203 may be positioned on the tube, such that material, exemplified by material 1204, 1205 and 1206, between the apertures 1201-1203 forms first and second struts. Two exemplary struts 1207 and 1208 are indicated in FIG. 12 by heavy dashed lines. As noted, a generally helical curve may include minor zigzags, not necessarily all the same, as exemplified by generally helical curves formed by the two exemplary struts 1207 and 1208. These zigzags are more clearly seen in the insert in FIG. 12, for example in struts 1209 and 1210, which are indicated by heavy solid and dashed lines.

FIG. 12 shows the expanded filter 1010 as the filter 1010 appears when mounted on an expanded housing 1000 (e.g., FIG. 11), although the housing 1000 is not shown in FIG. 12. A filter 1010 made of a shaped foil tube 1200 may be made of a polymer, such as PET or PU. The wall of the foil tube 1200 may be about 10 μm to about 100 μm thick, preferably about 15 μm to about 75 μm thick, and more preferably about 20 μm to about 50 μm thick. The thickness of the foil tube 1200 wall may decrease continuously in a distal direction in the tapered filter section 1140, such as a result of blow mold manufacturing.

The shape and size of the holes may differ in different parts of the expandable filter 1010. In the distal tubular filter section 1220, the holes, exemplified by hole 1221, may be longer (in a longitudinal direction) than wide (in a circumferential direction). The holes 1221 may be defined in circumferential rows. Holes 1221 in adjacent rows may be staggered in the circumferential direction, and partially overlap in the longitudinal and circumferential directions, as shown in FIG. 10. Such staggering and overlapping enables the distal tubular section 1220 to easily dilate during assembly, without requiring resilient stretching of the material. This dilation may facilitate inserting the impeller 290 into the housing 1000 through the distal end of the housing 1000. Furthermore, such staggering generally enables disposing the holes 1221 closer together and, therefore making the filter 1010 more transparent to blood flow. Each hole 1221 in the distal tubular filter section 1220 may have an enlarged portion located centrally in a longitudinal slot. After the insertion of the impeller 290 and the return of the distal tubular section 1220 to its normal diameter, the enlarged portion advantageously has a relatively large open contact area.

The expandable filter 1010 may further include a transitional zone 1222 where the distal tubular filter section 1220 and the tapered filter section 1140 meet. Holes, exemplified by hole 1223, in the transitional zone 1222 may be longer and wider than adjacent holes of the tapered filter section 1220. Preferably, the holes 1223 in the transitional zone 1222 may be at least twice as large as the adjacent holes, exemplified by hole 1224, in the tapered filter section 1140. In one embodiment, for each pair of circumferentially adjacent holes 1224 in a row of the tapered filter section 1140, the transitional zone 1222 has one hole 1223 that circumferentially straddles the two holes 1224. Thus, the number of holes in a circumferential row in the transitional zone 1222 may be half the number of holes in a circumferential row in the tapered filter section 1140. In some other embodiments, other ratios may be used, such as 3:1, 4:1 or 3:2. Each hole 1223 in the transitional zone 1222 may be about twice, thrice or another multiple as long (in the longitudinal direction) and about twice, thrice or another multiple as wide (in the circumferential direction) as the hole 1224 in the tapered filter section 1140, depending on the ratio of the number of holes 1224 in one row of the tapered filter section 1140 to the number of holes 1223 in one row of the transitional zone 1222.

The dimensions and shapes of the holes 1201-1203 and 1224 and dimensions of the struts 1207-1208 should be chosen such that, when the tapered filter section 1140 is fully open, the housing 1000 can be inserted into the tapered filter section 1140, without exceeding limits of elastic deformation of the material. For example, the length of two circumferentially adjacent struts 1207-1208 (on zigzag of a zigzag circumferential ring), multiplied by the number of apertures 1201-1203 in a circumferential row, should about equal the circumference of a fully expanded housing 1000, taking into account any local elastic deformation of the filter material.

Adjacent holes 1223 in the transitional zone 1222 are separated from each other by struts that are wider than an adjacent strut 1207-1208 of the tapered filter section 1140. These wider struts stabilize the larger holes 1223.

As can be seen in FIG. 12, the holes 1224 in a distal region of the tapered filter section 1140 are narrower, in a circumferential direction, than the holes 1201-1203 in a proximal region of the tapered filter section 1140. In other words, sizes of the apertures 1201-1203 increase monotonically in the proximal direction, along the longitudinal axis. In addition, in the distal tubular filter section 1220, the holes 1221 take the form of narrow axial slits, which are offset from each other in a circumferential direction. This is advantageous, as narrow holes can widen when the expandable filter 1010 is expanded at the distal tubular filter section 1220 and the distal region of the tapered filter section 1140, such as when the impeller 290 is inserted into the housing 1000. Wider holes are bounded by thicker struts, particularly in the tapered filter section 1140. The struts have a width of between about 30 μm in the distal region, and about 60 μm in the proximal region, of the tapered filter section 1140. Preferably, the largest diameter of the holes in the tapered filter section 1140 is between about 300 μm and about 500 μm.

In the embodiment shown in FIG. 12, the proximal tubular filter section 1225 has no holes. However, holes in the proximal tubular filter section 1225 may be desirable.

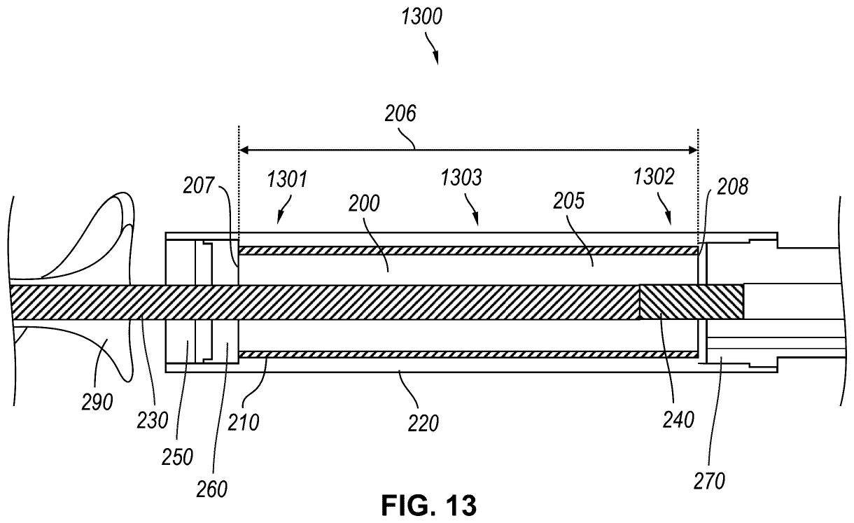

Referring to FIG. 13, in various aspects, a blood pump motor 1300 may be provided that has a magnet 205 with an opening extending from a distal end 207 to a proximal end 208. The opening may be filled with a distal shaft 230 and a proximal shaft 240. As disclosed herein, in some embodiments, at least a portion of the distal shaft may have a non-circular cross-section (such as a geometric shape having 3-10 sides, or having one or more recesses, protrusions, or both) when viewed along its central axis. In some embodiments, a single shaft extends through the magnet, but the shaft may include at least a portion having a non-circular cross-section. In some embodiments, the magnet may have distal end portion 1301, a proximal end portion 1302, and a rotationally symmetrical middle portion 1303 located between the distal end portion and the proximal end portion, the middle portion having a circular cross-section.

Various modifications may be made to the systems, methods, apparatus, mechanisms, techniques, and portions thereof described herein with respect to the various figures, such modifications being contemplated as being within the scope of the invention. For example, while a specific order of steps or arrangement of functional elements is presented in the various embodiments described herein, various other orders/arrangements of steps or functional elements may be utilized within the context of the various embodiments. Further, while modifications to embodiments may be discussed individually, various embodiments may use multiple modifications contemporaneously or in sequence, compound modifications and the like.

Although various embodiments which incorporate the teachings of the present invention have been shown and described in detail herein, those skilled in the art can readily devise many other varied embodiments that still incorporate these teachings. Thus, while the foregoing is directed to various embodiments of the present invention, other and further embodiments of the invention may be devised without departing from the basic scope thereof. As such, the appropriate scope of the invention is to be determined according to the claims.

1. A blood pump motor, comprising:
a rotor having a central axis of rotation, the rotor comprising a magnet having a length extending from a distal end to a proximal end; and
a stator disposed around at least a portion of the rotor;
wherein:
the rotor is free of an opening extending from the distal end to the proximal end, and/or
the magnet has distal end portion, a proximal end portion, and a rotationally symmetrical middle portion located between the distal end portion and the proximal end portion, the rotationally symmetrical middle portion having a circular cross-section.

2. The blood pump motor of claim 1, further comprising a housing disposed around the stator.

3. (canceled)

4. (canceled)

5. The blood pump motor of claim 1, wherein a diameter of the blood pump motor is less than 3.3 mm.

6. The blood pump motor of claim 1, wherein the proximal end portion and/or the distal end portion comprises a bore extending into an outer surface, or a protrusion extending away from an outer surface.

7. The blood pump motor of claim 6, wherein at least one bore or protrusion is configured to extend radially inward or outward from the outer surface.

8. The blood pump motor of claim 1, further comprising a distal shaft operably coupled to the distal end of the magnet, a proximal shaft operably coupled to the proximal end of the magnet, or both.

9. The blood pump motor of claim 8, further comprising a distal bore extending partially into a distal end of the magnet, a proximal bore extending partially into a proximal end of the magnet, or both.

10. The blood pump motor of claim 9, wherein the distal bore extends through the distal end portion and the rotationally symmetrical middle portion is free of the distal bore, the proximal bore extends through the proximal end portion and the rotationally symmetrical middle portion is free of the proximal bore, or both.

11. The blood pump motor of claim 9, wherein the distal bore forms a chamfered surface at the distal end of the magnet, the proximal bore forms a chamfered surface at the proximal end of the magnet, or both.

12. The blood pump motor of claim 9, wherein the distal shaft extends into the distal bore and the distal bore extends less than 3 mm into the magnet, the proximal shaft extends into the proximal bore and the proximal bore extends less than 3 mm into the magnet, or both.

13. (canceled)

14. (canceled)

15. (canceled)

16. The blood pump motor of claim 8, wherein the distal shaft is coupled to a distal shaft cap at a proximal end of the distal shaft, and/or the proximal shaft is coupled to a proximal shaft cap at a distal end of the proximal shaft.

17. The blood pump motor of claim 16, wherein the distal shaft cap and/or proximal shaft cap have an inner surface configured to be coupled to an outer surface of the magnet.

18. The blood pump motor of claim 17, wherein at least a portion of the inner surface of the distal shaft cap and/or proximal shaft cap has a non-circular cross-section when viewed along its central axis.

19. (canceled)

20. (canceled)

21. (canceled)

22. The blood pump motor of claim 8, wherein the distal shaft and/or proximal shaft is adhered to the magnet.

23. The blood pump motor of claim 8, wherein the distal shaft and/or proximal shaft is mechanically coupled to the magnet.

24. (canceled)

25. (canceled)

26. (canceled)

27. The blood pump motor of claim 8, wherein the distal shaft is the same length as the proximal shaft.

28. The blood pump motor of claim 8, wherein the distal shaft is shorter than the proximal shaft.

29. The blood pump motor of claim 1, wherein the magnet comprises an integral distal shaft extending from the distal end of the magnet, an integral proximal shaft operably coupled to the proximal end of the magnet, or both.

30. (canceled)

31. (canceled)

32. The blood pump motor of claim 8, further comprising a distal bearing disposed distal to the magnet, a proximal bearing disposed proximal to the magnet, or both.

33. (canceled)

34. (canceled)

35. (canceled)

36. A blood pump, comprising:
a motor section having a blood pump motor of claim 1;
a pump housing attached to the motor section and defining an input port and an output port; and
an impeller disposed within the pump housing and configured, when rotated, to pump blood from the input port to the output port, the impeller being operably coupled to the blood pump motor.

37-44. (canceled)
